(19) United States
(12) Patent Application Publication
(10) Pub. No.: US 2026/0215677 A1
(43) Pub. Date: Jul. 30, 2026

(54) IOL SELECTION SYSTEM AND METHOD

(71) Applicant: Alcon Inc., Fribourg (CH)

(72) Inventors: Sinchan Bhattacharya, Denton, TX (US); Bryan Stanfill, Mansfield, TX (US); Shruti Siva Kumar, Foothill Ranch, CA (US); Praneeth Kurpad Narayanamurthy, Arlington, TX (US); Ramesh Sarangapani, Coppell, TX (US)

(21) Appl. No.: 19/447,699

(22) Filed: Jan. 13, 2026

Related U.S. Application Data
(60) Provisional application No. 63/751,710, filed on Jan. 30, 2025.

(51) Int. Cl. A61B 3/00; A61B 3/103; A61F 2/16

(52) U.S. Cl. CPC A61B 3/0025; A61B 3/103; A61F 2/1613

(57) ABSTRACT

Intra-ocular lens (IOL) data describing an IOL model is processed using a machine learning model to obtain a selected calculator of a plurality of calculators that may be used to estimate the post-operative refractive error of the IOL model. The IOL data may be processed with patient data, such as eye measurements and patient history. Eye measurements may include anterior chamber depth (ACD) and white-to-white (WTW) distance. Patient history may include whether an eye has undergone past refractive error correction surgery. The selected calculator may be used to calculate a post-operative refractive error to guide selection of an IOL.

### TECHNICAL FIELD

The present disclosure relates generally to methods for the treatment of cataracts and, more particularly, to treatments including the use of intraocular lenses (IOL).

### BACKGROUND

Light received by the human eye passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light.

Through age or disease, the crystalline lens may become cloudy, a condition known as a cataract. Cataracts are a readily treated by removing the crystalline lens and inserting an artificial lens, known as an intraocular lens (IOL). The IOL may be fabricated to additionally correct for aberrations of the patient's eye, such as spherical error and astigmatism. If the optical properties of the IOL are not correct, the patient's eye will have post-operative refractive error.

It would be an advancement in the art to facilitate the accurate selection of an IOL to reduce post-operative refractive error.

### BRIEF SUMMARY

The present disclosure relates generally to a system for predicting post-operative refractive error of an eye following placement of an IOL.

In one aspect, a method for intra ocular lens (IOL) selection includes: receiving, by a computing device, measurements of an eye of a patient; receiving, by the computing device, IOL data describing an IOL model; processing, by the computing device, the measurements and the IOL data with a machine learning model to obtain a selected calculator of a plurality of calculators, each calculator of the plurality of calculators; processing, by the computing device, the measurements and the IOL data using the selected calculator to obtain an estimated post-operative refractive error for the IOL model; and generating, by the computing device, an output according to the estimated post-operative refractive error.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of a human eye.

FIG. 2 illustrates an example intraocular lens (IOL).

FIG. 3 illustrates an approach for training a machine learning model to assign scores to calculators for estimating post-operative refractive error of an eye having an implanted IOL, in accordance with certain embodiments.

FIG. 4 illustrates a system for selecting an IOL to implant in an eye, in accordance with certain embodiments.

FIG. 5 illustrates a method for selecting an IOL, in accordance with certain embodiments.

FIG. 6 illustrates an example computing device that implements, at least partly, one or more functionalities for performing post-operative evaluation of placement of an IOL, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Particular embodiments of the present disclosure provide a machine learning model to facilitate estimation of post-operative refractive error of an eye following placement of an IOL. In particular, the machine learning model may be used to select a calculator from a plurality of calculators for calculating the post-operative refractive error.

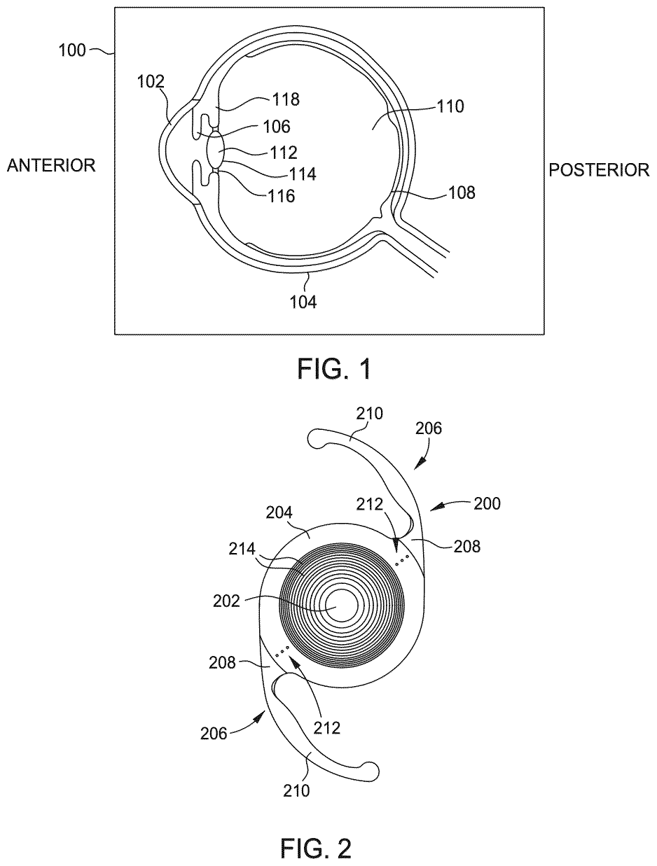

FIG. 1 is a diagram illustrating parts of the human eye 100 that may be understood with respect to the anterior side, through which light enters the eye 100, and the posterior side opposite the anterior side. At the anterior side of the eye 100, a thin transparent layer known as the cornea 102 is linked to the sclera 104, which forms the generally spherical wall of the eye 100. The cornea 102 and sclera 104 are connected by a ring called the limbus. The iris 106, the color of the eye 100, and an opening defined by it, the pupil, are positioned behind the cornea and are visible due to the cornea's 102 transparency. The retina 108 is formed on an interior surface of the sclera 104 opposite the cornea 102 and iris 106. The volume defined by the sclera 104 is occupied by the transparent jelly of the vitreous body 110.

The crystalline lens 112 is a transparent, biconvex structure in the eye 100 that, along with the cornea 102, helps to refract light to be focused on the retina 108. The lens 112, by changing its shape, functions to change the focal distance of the eye 100 so that it can focus on objects at various distances, thus allowing a sharp real image of the object of interest to be formed on the retina 108. This adjustment of the lens 112 is known as accommodation, and is similar to the focusing of a photographic camera via movement of its lenses.

The lens 112 is positioned behind the iris 106 in a capsular bag 114. The capsular bag 114 is attached at its perimeter to the suspensory ciliary ligament 116. The ciliary ligament 116 attaches the capsular bag 114 to the ciliary body 118. The ciliary body 118 is a ring-shaped muscle that attaches the ciliary ligament 116 to the sclera 104 and which can contract or relax in order to change the shape of the lens 112.

Various diseases and disorders of the lens 112 may be treated with an IOL. By way of example, not necessarily limitation, an IOL according to embodiments of the present disclosure may be used to treat cataracts, large optical errors in myopic (near-sighted), hyperopic (far-sighted), and astigmatic eyes 100, ectopia lentis, aphakia, pseudophakia, and nuclear sclerosis. However, for purposes of description, the IOL embodiments of the present disclosure are described with reference to cataracts, which often occurs in the elderly population.

FIG. 2 illustrates an example IOL 200. The toric IOL 200 includes a lens portion 202 that focuses light passing through the iris 106 onto the retina 108. The lens portion 202 may be surrounded by a peripheral ring 204 that is not used to focus light. Two or more haptics 206 may secure to the peripheral ring 204. Each haptic 206 may include a base 208 secured to the peripheral ring 204 and extending outwardly therefrom. A spring arm 210 secures to the base and extends both outwardly from the peripheral ring 204 and circumferentially around the peripheral ring 204. In use, the spring arms 210 push outwardly against the capsular bag 114 and hold the toric IOL 200 in a desired position.

Marks 212 may be formed on the peripheral ring 204. The marks 212 facilitate alignment of the IOL with the eye 100 of the patient. The marks 212 in the illustrated toric IOL 200 include two sets of dots (e.g., depressions or bumps), such as circular dots, formed on the peripheral ring 204 opposite one another. For example, each set may include, two, three, or more dots. The dots of each set may be collinear with one another and be collinear with the dots of the other set. Some toric IOLs 200 are multi-focal. The lens portion 202 may include rings 214 that define the boundary between regions of the lens portion 202 with different focal lengths.

FIG. 2 is one example of an IOL that may be used according to the approach described herein. An IOL according to any of the embodiments described herein may be of one or more of the following types:
A toric IOL
A monofocal IOL
A multifocal IOL
An extended depth of focus IOL
Light-Adjustable IOL
Phakic IOL
Aspheric IOL

Each of the above types of IOL may be available in a plurality of configurations defined by parameters. Examples of such parameters may include:
Spherical power
Cylindrical power
Toric axis
Near and/or intermediate add powers (for multifocal IOLs)

The above-listed examples of IOL types and parameters of IOLs is exemplary only. Any type of IOL and any parameters may be used as an input to the system and method described below.

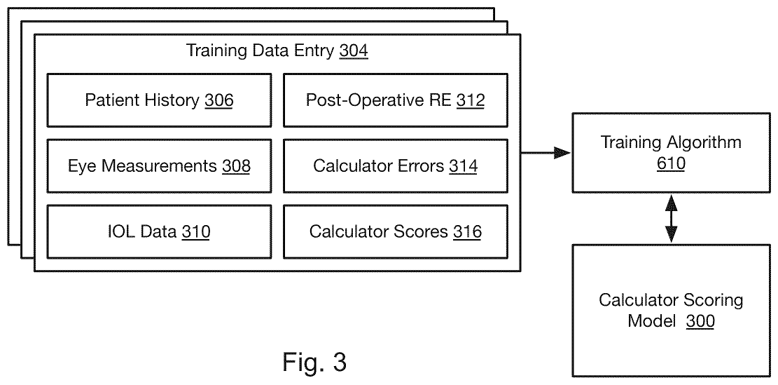

FIG. 3 illustrates an approach for training a calculator scoring model 300 to predict the error of calculators used to calculate the post-operative refractive error of an eye 100 following placement of an IOL. As discussed in greater detail below, different calculators provide different accuracy in different situations. The calculator scoring model 300 may therefore be trained to select a calculator to account for this variation in accuracy.

Examples of calculators may include the ALCON artificial intelligence power calculator, SRK/T calculator, HAIGIS calculator, BARRET UNIVERSAL II calculator, RT2D calculator, or any other calculator. The approach described herein may be used with any calculator. In particular, a definition of the input arguments of the calculator and the calculator itself may be used according to the approach described below without regard to the actual internal function of the calculator.

Input arguments to a calculator may include the type of IOL, one or more parameters describing the IOL, pre-operative measurements and/or images of the eye 100, intra-operative measurements of the eye 100 (e.g., following phacoemulsification), patient history, or other information.

Examples of measurements may include some or all of:
Pre-operative spherical error
Pre-operative cylindrical error
Pre-operative cylindrical error axis
Intra-operative spherical error
Intra-operative cylindrical error
Intra-operative cylindrical error axis
Anterior chamber depth
White-to-white (WTW) (e.g., distance across cornea)
Optical coherence tomography (OCT) image(s) of the eye 100
Ultrasound biomicroscopy (UBM) video(s) of the eye 100
Digital models of the eye 100, IOL or a combination of the two

Examples of patient history information that may be relevant may include:
Whether the patient has had refractive error-correction surgery and, if so, which type (e.g., laser-assisted in-situ keratomileusis (LASIK), photorefractive keratectomy (PRK), small incision lenticular extraction (SMILE), or the like).
Whether prior refractive error-correction surgery was to correct myopia or hyperopia.

The calculator scoring model 300 may be trained by a training algorithm 302 using a plurality of training data entries 304. Each training data entry 304 may represent one eye 100 of a patient that has had an IOL implanted therein. Each training data entry may include a patient history 306 (e.g., a patient history as defined above), eye 100 measurements 308 (e.g., any of the pre-or intra-operative eye 100 measurements listed above), and IOL data 310 (e.g., a type and parameters of the implanted IOL).

Each training data entry 304 may also include a post-operative refractive error 312 of the eye 100, e.g., spherical error, cylindrical error, cylindrical axis. Post-operative refractive error 312 may further include a measure of presbyopia, such as a near point measurement or an add power. The post-operative refractive error 312 may be measured following implantation of the IOL and following healing of the eye 100, such as one, two, three, or more weeks following implantation.

Each training data entry 304 may further include calculator errors 314. Each calculator error 314 may include an identifier of a calculator, an error magnitude, and possibly an error sign. The calculator error 314 for a calculator may be calculated by inputting input arguments to the calculator (e.g., any of the patient history 306, eye 100 measurements 308, and/or IOL data 310 that the calculator uses as input arguments), receiving a refractive error estimate, and calculating the calculator error as a difference between the refractive error estimate and the post-operative refractive error 312. The calculator errors may be calculated with respect to spherical error, cylindrical error, cylindrical error axis, presbyopia, or any other refractive error.

Each training data entry 304 may also include a calculator score 316 for each calculator. For example, the calculator scores 316 may be derived from the calculator errors 314, such as by normalizing, scaling, or otherwise processing the calculator errors 314. A calculator score 316 may be a single value whereas the calculator errors 314 may include multiple values. Accordingly, the calculator score 316 for a calculator may be calculated as a combination of the magnitudes (and possibly signs) of the spherical error, cylindrical error, cylindrical error axis, presbyopia, or any other refractive error metric included in the calculator error 314 for that calculator, such as by adding, weighting and adding, or some other function.

In some embodiments, the calculator scoring model 300 is trained by processing inputs from the training data entry 304, e.g., data that is available pre-or intra-operatively such as the patient history 306, eye 100 measurements 308, and IOL data 310 to obtain an estimated calculator score for each calculator of a plurality of calculators. The training algorithm then compares the estimated calculator score for each calculator with the calculator score 316 for that calculator and updates the calculator scoring model 300 according to the comparisons.

Following training with an initial set of training data entries 304, the calculator scoring model 300 may continue to be trained with new training data entries 304 that become available. In addition, the calculator scoring model 300 may be retrained to handle additional calculators. For example, the calculator errors 314 and calculator scores 316 of the training data entries may be calculated for the new calculator and the calculator scoring model 300 may be further trained with the updated training data entries 304 or completely new training data entries that include calculator errors 314 and calculator scores 316. The calculator scoring model 300 may be incorporated into a machine learning operation (MLOps) framework that may monitor performance of the calculator scoring model 300 and generate alerts or invoke retraining if performance of the calculator scoring model 300 drops.

Note that the calculator errors 314 are an intermediate result and therefore may be omitted from the training data entry 304 in some embodiments. Likewise, the post-operative refractive error 312 is an intermediate result and may be omitted. However, including the post-operative refractive error 312 enables the training data entry 304 to be expanded to reference additional calculators as the additional calculators become available.

The calculator scoring model 300 may be implemented as a decision tree, with each node of the tree corresponding to an input, e.g., an attribute included in one or more of the patient history 306, eye measurements, 308 and IOL data 310. The calculator scoring model 300 may be implemented as a random forest, long short term memory (LSTM), generative adversarial model (GAN), a neural network, deep neural network (DNN), convolution neural network (CNN), recurrent neural network (RNN), Bayesian network, genetic algorithm, logistic regression model, multiple linear regression model, multivariate polynomial regression model, support vector regression model, or any other type of machine learning model.

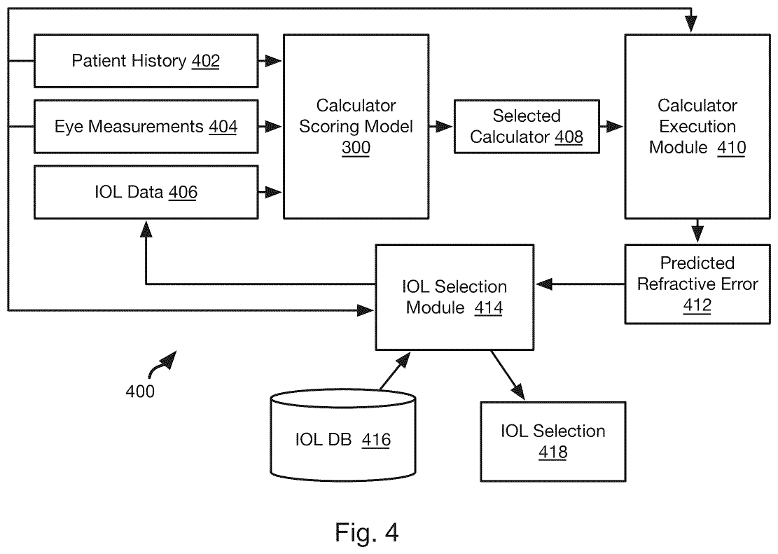

FIG. 4 illustrates a system 400 that uses the calculator scoring model 300 to select an IOL for an eye 100 of a patient. The system 400 may input a patient history 402, eye measurements 404, and IOL data 406 for an eye 100 of the patient to the calculator scoring model 300. The calculator having the lowest (a lower value indicating higher accuracy in this example) score as determined by the calculator scoring model 300 may then be selected as a selected calculator 408. A calculator execution module 410 may then calculate a predicted refractive error 412 using the selected calculator 408. The calculator execution module 410 may use whichever of the patient history 402, eye measurements 404, and IOL data 406 is defined as input arguments for the selected calculator 408 and execute the selected calculator 408 with respect to the input arguments to obtain the predicted refractive error 412.

The IOL data 406 that is input to the calculator scoring model 300 may be selected by a human expert. In some other embodiments, an IOL selection module 414 may access a database 416 including entries for a plurality of IOL models and including IOL data for each model. As used herein an “IOL model” is a design of an IOL offered by a manufacturer, e.g., as designated by a model name and having a type and nominal values for some or all of the parameters defining an IOL as outlined above. The entry for an IOL model may list information indicating the appropriate circumstance for using the IOL model, e.g., eye measurements and/or patient history values that are compatible with that IOL model. The IOL selection module 414 may therefore make an initial selection of an IOL model from the IOL database 416 based on the patient history 402 and/or eye measurements 404 and input the IOL data 406 for that IOL model to the calculator scoring model 300.

The IOL selection module 414 may select two or more IOL models from the IOL database 416 that are compatible with the patient history 402 and/or eye measurements 404 and process their corresponding IOL data 406 with the calculator scoring model 300 to obtain corresponding predicted refractive errors 412. The IOL selection module 414 may output an IOL selection 418, e.g., an identifier of the IOL model having the lowest predicted refractive error 412.

The process of selecting IOL models to process with the calculator scoring model 300 may also be performed by a human operator.

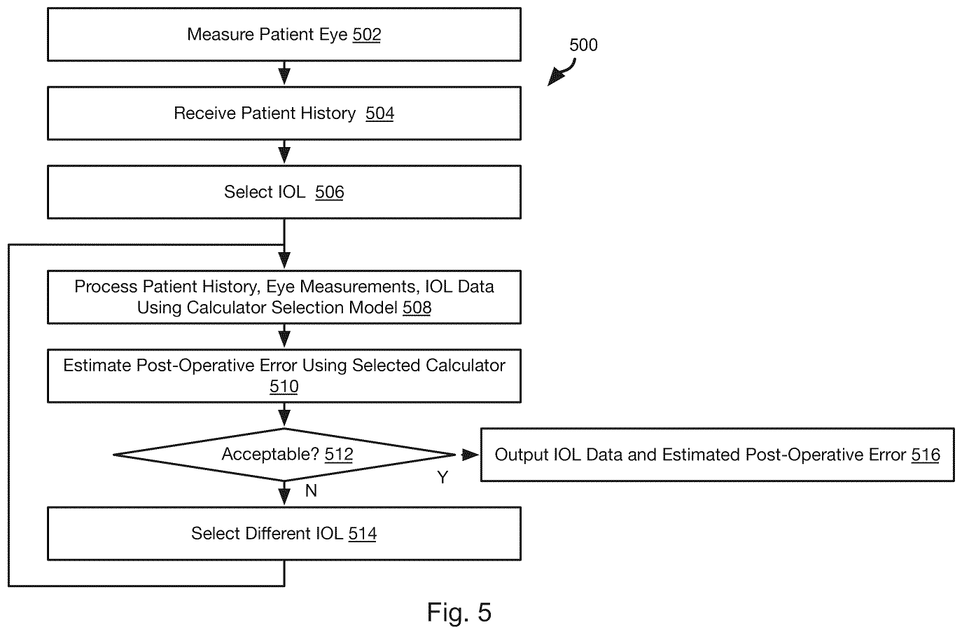

FIG. 5 illustrates a method 500 that may be performed using the calculator scoring model 300 with respect to an eye 100 of a patient in order to select an IOL to implant in the eye 100. The method 500 includes measuring, at step 502, the eye 100 of a patient to obtain eye measurements as defined above. Step 502 may be performed using one or more optical imaging modalities, such as an autorefractor, aberrometer, optical coherence tomography (OCT) device, corneal topography device, keratometry device, three-dimensional camera, or other device. Step 502 includes performing a subjective optometry exam. Step 502 may include one or both of pre-operative measurements and intra-operative measurements, e.g., following phacoemulsification and prior to placement of an IOL.

The method 500 includes receiving, at step 504, a patient history for the eye 100, such as a patient history as defined above. The method 500 includes selecting, at step 506, an IOL. Step 506 may be performed by a human or the IOL selection module 414. The IOL may be selected as suitable for the geometry of the eye 100 and the refractive error of the eye 100, e.g., of the cornea 102.

The method 500 includes processing, at step 508, the patient history, eye measurements, and IOL data for the IOL selected at step 506 with the calculator scoring model 300 to obtain a selected calculator, e.g., the calculator having the score indicating highest accuracy in the output of the calculator scoring model 300. An estimated post-operative refractive error for the IOL data may be estimated using the selected calculator at step 510.

The IOL selection module 414 or a human operator may evaluate, at step 512 whether the estimated post-operative refractive error is acceptable, e.g., whether each of the spherical error and cylindrical error are below an acceptable threshold, e.g., 0.25 Diopters. If not, the human operator or IOL selection module 414 may select, at step 514, a different IOL model and process the IOL data for that IOL model at step 508 as described above.

If the estimated post-operative refractive error calculated at step 510 for an IOL model is found to be acceptable by a human operator or the IOL selection module 414, the method 500 may include outputting, at step 516, information, such as an identifier of the IOL model, the IOL data, the estimated post-operative refractive error, and/or other information. Step 516 may include outputting information to a display device, sending the information in an email, storing the information in a storage device for later retrieval, or some other output modality.

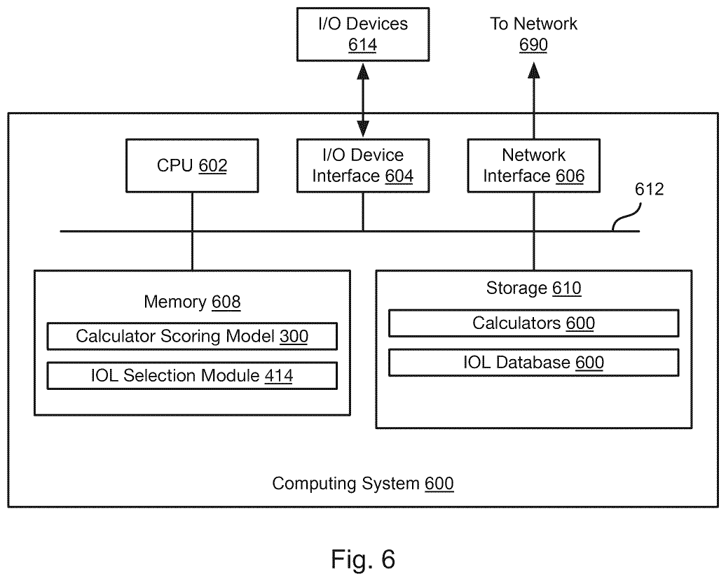

FIG. 6 illustrates an example computing system 600 that may be used to implement the system and method described herein. Note that different computing devices may be used for training and utilization of the calculator scoring model 300.

As shown, computing system 600 includes a central processing unit (CPU) 602, one or more I/O device interfaces 604, which may allow for the connection of various I/O devices 614 (e.g., keyboards, displays, mouse devices, pen input, etc.) to computing system 600, network interface 606 through which computing system 600 is connected to network 690, a memory 608, storage 610, and an interconnect 612.

CPU 602 may retrieve and execute programming instructions stored in the memory 608. Similarly, CPU 602 may retrieve and store application data residing in the memory 608. The interconnect 612 transmits programming instructions and application data, among CPU 602, I/O device interface 604, network interface 606, memory 608, and storage 610. CPU 602 is included to be representative of a single CPU, multiple CPUs, a single CPU having multiple processing cores, and the like.

Memory 608 is representative of a volatile memory, such as a random access memory, and/or a nonvolatile memory, such as nonvolatile random access memory, phase change random access memory, or the like. As shown, memory 608 may store the calculator scoring model 300, the IOL selection module 414, and any other executable code for training the calculator scoring model 300 and/or utilizing the calculator scoring model 300.

The computing system 600 may include storage 610, which may be non-volatile memory, such as a disk drive, solid state drive, or a collection of storage devices distributed across multiple storage systems.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for intra ocular lens (IOL) selection, the method comprising:
receiving, by a computing device, measurements of an eye of a patient;
receiving, by the computing device, IOL data describing an IOL model;
processing, by the computing device, the measurements and the IOL data with a machine learning model to obtain a selected calculator of a plurality of calculators, each calculator of the plurality of calculators;
processing, by the computing device, the measurements and the IOL data using the selected calculator to obtain an estimated post-operative refractive error for the IOL model; and
generating, by the computing device, an output according to the estimated post-operative refractive error.

2. The method of claim 1, wherein the measurements of the eye of the patient include an anterior chamber depth (ACD).

3. The method of claim 1, wherein the measurements of the eye of the patient include a white-to-white (WTW) distance of the eye of the patient.

4. The method of claim 1, wherein the measurements of the eye of the patient include a refractive error of the eye of the patient.

5. The method of claim 1, wherein the measurements of the eye of the patient include all of an anterior chamber depth (ACD), white-to-white (WTW) distance, and refractive error of the eye of the patient.

6. The method of claim 1, wherein the IOL data indicates a type of the IOL model.

7. The method of claim 6, wherein the type of the IOL model is one of toric, monofocal, multifocal, extended depth of focus, light adjustable, and phakic.

8. The method of claim 7, wherein the IOL data includes a spherical power, a cylindrical power, and a toric axis.

9. The method of claim 1, further comprising receiving a history of the eye of the patient and processing the history using the machine learning model.

10. The method of claim 9, wherein the history indicates whether the eye has undergone refractive error correction surgery.

11. A system for intra ocular lens (IOL) selection, the system comprising:
a computing device including one or more processing devices and one or more memory devices operably coupled to the one or more processing devices, the one or more memory devices storing executable code that, when executed by the one or more processing devices, causes the one or more processing devices to:
receive measurements of an eye of a patient;
receive IOL data describing an IOL model;
process the measurements and the IOL data with a machine learning model to select a selected calculator from a plurality of calculators;
process the measurements and the IOL data using the selected calculator to obtain an estimated post-operative refractive error for the IOL model; and
generate an output according to the estimated post-operative refractive error.

12. The system of claim 11, wherein the measurements of the eye of the patient include an anterior chamber depth (ACD).

13. The system of claim 11, wherein the measurements of the eye of the patient include a white-to-white (WTW) distance of the eye of the patient.

14. The system of claim 11, wherein the measurements of the eye of the patient include a refractive error of the eye of the patient.

15. The system of claim 11, wherein the measurements of the eye of the patient include all of an anterior chamber depth (ACD), white-to-white (WTW) distance, and refractive error of the eye of the patient.

16. The system of claim 11, wherein the IOL data indicates a type of the IOL model.

17. The system of claim 16, wherein the type of the IOL model is one of toric, monofocal, multifocal, extended depth of focus, light adjustable, and phakic.

18. The system of claim 17, wherein the IOL data includes a spherical power, a cylindrical power, and a toric axis.

19. The system of claim 11, wherein the executable code, when executed by the one or more processing devices, further causes the one or more processing devices to receive a history of the eye of the patient and process the history using the machine learning model.

20. The system of claim 19, wherein the history indicates whether the eye has undergone refractive error correction surgery.
